(19) United States
(12) Patent Application Publication
(10) Pub. No.: US 2004/0106864 A1
(43) Pub. Date: Jun. 3, 2004

(54) Method of predicting stroke evolution utilising mri

(76) Inventors: Stephen Edward Rose, Bunya, Queensland (AU); Mark Philip Griffin, Corinda Queensland (AU); Andrew Lindsay Janke, St. Lucia (AU); Jonathan Brandon Chalk, Ashgrove Queensland (AU); Geoffrey John McLachlan, Indooroopilly Queensland (AU); David Peel, Middle Park Queensland (AU); Deming Wang, Indooroopilly (AU)

Correspondence Address: NIXON & VANDERHYE, PC, 1100 N GLEBE ROAD, 8TH FLOOR, ARLINGTON, VA, 22201-4714, US

(21) Appl. No.: 10/471,082

(22) PCT Filed: Mar. 6, 2002
(86) PCT No.: PCT/AU02/00256

(30) Foreign Application Priority Data
Mar. 7, 2001 (AU) PR 3587

(51) Int. Cl. A61B 5/05

(52) U.S. Cl. 600/410

(57) ABSTRACT

A method predicting stroke evolution uses magnetic resonance diffusion and perfusion images obtained shortly after the onset of stroke symptoms to automatically estimate the eventual volume of dead cerebral tissue resulting from the stroke. The diffusion and perfusion images are processed to extract region(s) of interest presenting tissue at risk of infarction. A midplane algorithm is also used to calculate ratio and diffusion and perfusion measures for modelling infarct evolution. A parametric normal classifier algorithm is used to predict infarct growth using the calculated measures.

THIS INVENTION relates to a method for predicting infarct evolution using magnetic resonance imaging (MRI) and image processing. In particular, the invention is directed to an automated method for estimating the volume of dead nervous tissue resulting from a stroke, using imaging information obtained shortly after the onset of stroke symptoms.

### BACKGROUND ART

Typically, a person suffers an ischemic infarction or stroke when a blood vessel is blocked, causing cerebral nervous tissue to be deprived of oxygen. In the initial few hours after a stroke, there is usually a significantly reduced blood supply to a region of nervous tissue due to a blocked or nearly-blocked blood vessel which would otherwise supply oxygen to that tissue. The nervous tissue deprived of adequate blood supply does not necessarily die immediately. It can often die over the next 18 hours or so. The prediction of the final size of the stroke, i.e. the final volume of dead tissue is very difficult.

If the stroke evolution is known, the patient can receive appropriate treatment. For example, if the stroke is expected to evolve into a significant volume of dead nervous tissue, the patient can be placed in intensive care and/or administered strong medication in an effort to minimise the effects of the stroke. Alternatively, if the stroke is not expected to evolve further, the patient may be given less intensive therapy, and avoid the side effects associated with the powerful drugs. An ability to predict or estimate stroke evolution would therefore be a highly beneficial and useful tool in the treatment of stroke patients.

Known methods of stroke evaluation generally rely on the use of subjective measures such as operator defined regions of interest on diffusion and perfusion maps to enable prediction of infarct size. However, these methods are time consuming to implement and require highly skilled practitioners. Further, there is a limited time window of opportunity for the administration of thrombolytic or neuroprotective therapy. Thus a basic criterion for a predictive model-based prognostic aid in the acute stroke clinic is that the method is both rapid and automated, or at least semi-automated.

There are so-called automated methods of predicting ischemic events or risk, but these are generally limited to cardiac infarctions. For example, U.S. Pat. No. 4,492,753 describes a method for determining the risk of future cardiac ischemic events based on measured protein levels in the patient blood plasma. U.S. Pat. No. 4,957,115 describes a device for determining the probability of death of cardiac patients based on analysis of electrode cardiograph waveforms. U.S. Pat. No. 5,276,612 describes a risk management system for cardiac patients which is also based on electrocardiograph measurements. Hitherto, there has been no satisfactory automated or semi-automated method of predicting stroke evolution.

It is an object of this invention to provide a method of predicting stroke evolution.

### SUMMARY OF THE INVENTION

This invention provides a model for predicting the evolution of stroke in humans, utilising diffusion and perfusion magnetic resonance images acquired in the acute phase of stroke. The predicted outcome can then be used to clinically guide therapeutic intervention to the stroke patients and/or evaluate the efficacy of novel stroke compounds in clinical drug trials.

The method involves:

(i) automatic extraction of regions-of-interest (ROIs) defining the ischemic lesion on diffusion weighted magnetic resonance images and regions of abnormal hemodynamic function on perfusion weighted magnetic resonance images. [These brain regions represent tissue-at-risk of infarction]

(ii) modeling the diffusion and perfusion parameters described within the bounds of hemodynamic abnormality to predict infarct growth.

More preferably, the method involves the steps of:

(i) automated extraction of brain regions which present tissue-at-risk of infarction, (ii) use of a mid-plane algorithm to calculate ratio :and diffusion and perfusion measures for modeling infarct evolution, and (iii) use of a parametric normal classifier algorithm to predict infarct growth.

In one form, the invention can be said to provide a method of predicting deterioration of cerebral tissue of a patient due to a stroke, the method including the steps of:

processing diffusion and perfusion images of the cerebral tissue obtained by magnetic resonance imaging shortly after the onset of stroke symptoms, to automatically define regions of interest on the images and to calculate diffusion and perfusion ratio measures, and

identifying pixels in the regions of interest representing tissue expected to go into infarction, by applying a classifier algorithm which uses a plurality of parameters including the calculated diffusion and perfusion ratio measures.

Other features and advantages of the invention will be apparent from the description of the preferred embodiment herein.

In order that the invention may be more fully understood and put into practice, a preferred embodiment thereof will now be described, by way of example only, with reference to the accompanying drawings.

### BRIEF DESCRIPTION OF THE DRAWINGS

FIG. 1 contains images representing the automated extraction of a diffusion lesion and MTT ROI. From top left to top right, (A) the isotropically weighted diffusion image, (B) the corresponding registered MTT map and (C) the composite MTT map derived from the product of the initial diffusion image, MTT mask and difference MTT map. The bottom images represent (D) the binary image of the composite MTT map, (E) the binary diffusion mask and (F) the binary MTT mask extracted after initial seeding from the diffusion mask and application of the 3D region growing algorithm.

FIG. 2 contains representative histograms plotting isotropically weighted diffusion pixel intensity versus MTT measures, and illustrates the modelling and classification functions. From top left to top right, the histograms for all penumbral pixels which correspond to tissue which survived the ischemic event (A) and those within the final infarcted lesion volume (B) for a given patient are presented. The true group allocation is shown in (C), where each point is classified into surviving or infarcted based upon the histograms (A) and (B). Bottom, left to right, (D) and (E) contain the normal functions modeling the frequency distributions of A and B, and (F) shows the predicted group allocation based on these frequency distributions.

FIG. 3 contains diffusion and perfusion images acquired for a representative patient from the training data sets (patient 10) of the example described herein, two hours after onset of symptoms. Top, left to right, (A) the DWI scan showing a poorly defined diffusion lesion in the deep white matter in the left hemisphere, (B) the MRA showing occlusion of the left MCA, (C) the MTT map with the extracted MTT mask highlighted and (D) the composite MTT map. Bottom left to right, (E) CBF, (F) CBV, (G) the follow-up T2-weighted scan (b=0) with predicted lesion highlighted and (H) the final lesion volume derived by subtraction of the initial T2 image from the follow-up scan.

FIG. 4 contains diffusion and perfusion images acquired for a representative patient from the validation data sets (patient 17) of the example described herein, ten hours after onset of symptoms. Top left to right, (A) the DWI scan showing a diffusion lesion in the left MCA territory (diffusion mask highlighted), (B) the MRA showing occlusion of the left MCA, and (C) the MTT map with extracted MTT mask highlighted. Bottom left to right, (D) CBF, (E) CBV and (F) the follow-up T2-weighted scan (b=0) with predicted lesion highlighted.

### DESCRIPTION OF PREFERRED EMBODIMENT

The method of predicting stroke evolution according to the preferred embodiment involves the computerised processing of brain scan images obtained shortly after the onset of stroke symptoms.

First, input magnetic resonance diffusion and perfusion images are acquired in the acute phase of stroke. Appropriate diffusion images can be acquired with standard diffusion- weighted MRI sequences¹ or diffusion tensor imaging (DTI) methods.² The methodology of the preferred embodiment of this invention has been developed to process isotropically weighted diffusion images (DWI) generated from diffusion tensor images by the method of Sorensen et al.³ However, the method would be applicable to process standard diffusion-weighted images where the lesion appears hyperintense⁴ or images of the apparent diffusion coefficient of water (ADC).^{5,6}

Perfusion images are defined as maps of cerebral blood flow (CBF), cerebral blood volume (CBV) and mean transit time (MTT) derived using dynamic susceptibility contrast imaging as described by stergaard.^{7,8} Absolute measures of CBF and CBV were calculated using the method of stergaard.⁹

Secondly, to enable registration of perfusion maps to diffusion images, raw spin-echo EPI perfusion images are then coregistered to the initial T2- weighted diffusion scan using a 6 parameter rigid body transformations In this case, the T2- weighted diffusion scan refers to a diffusion scan acquired without any diffusion encoding gradients.

Thirdly, after image registration, regions of abnormal hemodynamic function on MTT maps, which present tissue-at-risk of infarction, and lesions on diffusion images are automatically defined. This is achieved using an automated mid-plane algorithm to generate difference diffusion (dDWI) and difference perfusion (dMTT) images. Difference images refer to images generated by the subtraction of pixels in the contralateral hemisphere from corresponding pixels in the infarcted hemisphere. The mid-plane algorithm also allows calculation of diffusion and perfusion ratio measures used for modeling infarct evolution. In this case, ratio measures are calculated by dividing the intensity of each pixel within the region defined as tissue-at-risk of infarction by the corresponding pixel in the contralateral side.

The mid-plane algorithm involves flipping the image in the Y plane followed by registration of the mirrored image to its original form with a six parameter rigid body transformations. The mid-plane is then determined by halving the resulting rotations and translations. To aid delineation of the diffusion abnormality on the diffusion image, a composite image is calculated from the product of the initial diffusion image and the dDWI map on a pixel-by-pixel basis. A bimodal t-test is then performed on this image to create a binary diffusion mask.

In a similar fashion, a MTT composite image is calculated by multiplication of the initial MTT map with the dMTT map and with the diffusion weighted image on a pixel-by-pixel basis. This yields a MTT mask that is specific only to brain tissue as defined on the DWI scan.

Using the DWI mask as the initial seed, a three dimensional region-growing technique¹¹ is then employed to extract the MTT ROI from the composite MTT map. The extracted MTT ROI now defines the tissue-at-risk of infarction.

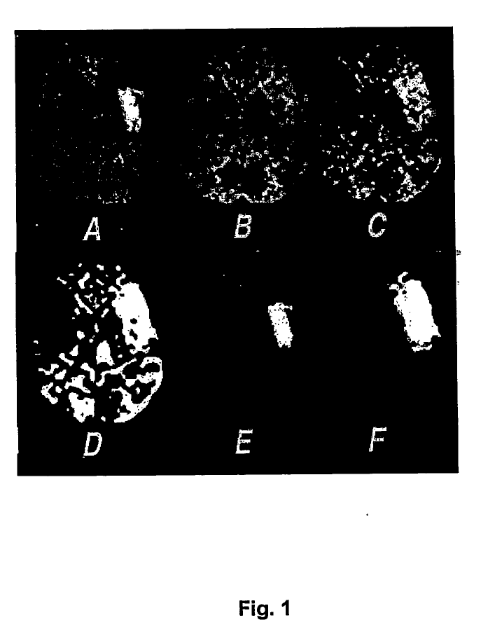

The task of extracting the MTT mask is simplified by only interrogating the hemisphere containing the ischemic lesion. Intermediate composite maps along with binary diffusion and MTT masks for a representative patient are given in FIG. 1. As all perfusion maps are coregistered, both absolute and ratio measures of CBF and CBV for each pixel within the bounds of MTT ROI can be calculated.

Fourthly, parametric normal classifiers¹² are employed to predict the spatial location and size of the final lesion from diffusion and perfusion parameters derived from the MTT ROI defining the tissue-at-risk of infarction. Diffusion and perfusion measures for each pixel within this region are calculated. In this embodiment of the invention, a classifier algorithm uses an eight-parameter vector (DWI, raDWI, CBF, raCBF, CBV, raCBV, MTT and raMTT) where ra denotes ratio measure between the ischemic and contralateral hemisphere. This algorithm enables classification of each pixel within the ROI defining the tissue-at-risk of infarction to either of two groups. Those pixels, which represent tissue destined to go onto infarction, and those representing tissue that will survive the ischemic event.

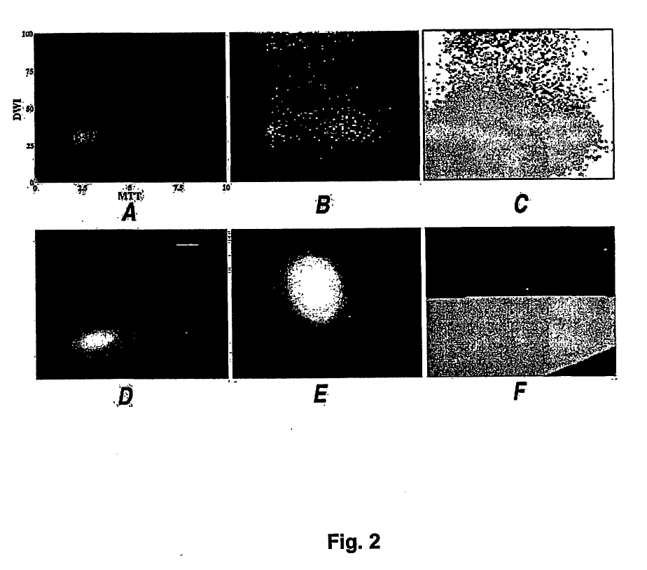

For the purpose of illustration, a model employing a parameter vector x containing the diffusion and MTT pixel intensities is defined (see FIG. 2). Representative frequency histograms are produced in which the isotropically weighted diffusion pixel intensity (arbitrary units) is plotted versus MTT measures for the surviving (FIG. A) and infarcted (FIG. B) pixels from a single patient. (Note that pixels can be classified as either surviving or infarcted from the patient's T2 scan taken a number of days after the onset of the stroke⁶) In order to produce these histograms the parameter space (DWI versus MTT) was divided into a number of bins. The pixels from a particular patient were allotted to bins based upon the value of their DWI and MTT parameters. Each bin was then colour coded where the lighter the bin the greater the number of pixels contained in the bin.

In FIG. 2(C), each histogram bin is classified into one of the two groups in accordance with the frequencies in the histograms A and B of FIG. 2. For each bin the number of pixels classified as surviving or infarcted were compared. Those bins with more surviving pixels were coloured grey, those with more infarcted pixels coloured black, and those with no pixels coloured white.

For ease of mathematical computation the histograms A and B were represented using normal distributions. This involved determining the mean and covariance matrix, in addition to counting the number of observations in each group. The normal distribution (ƒ) of the ith group can be expressed mathematically¹² as:

$${f}_{ i} ( x ) = \frac{1}{{( 2 π )}^{d / 2} {\vert {\sum }_{i} \vert}^{1 / 2}} exp ( - \frac{1}{2} {( x - {μ}_{i} )}^{T} {\sum }_{i}^{- 1} ( x - {μ}_{i} ) ) \qquad ( 1 )$$

where μᵢ denotes the mean parameter vector, Σᵢ the covariance matrix, and d the number of elements within the parameter vector. The prior probability (pᵢ) is the probability that a pixel chosen at random will belong to the Rh group, and is calculated by the number of pixels in the ith group divided by the total number of pixels over all of the groups. The histograms A and B given in FIG. 2, are modelled by the normal distributions in histograms D and E, respectively. These normal distributions are plotted so that the brighter the intensity the larger the value of ƒᵢ at that point.

The model classifies each new pixel according to the two normal distributions. Again for each point the relative heights of the two distributions are compared (see FIG. 2(F)). Those points where.,the surviving distribution is higher than the infarcted distribution are classified as surviving and shown in grey. The remaining points are classified as infarcted and shown in black.

In general this type of modelling strategy starts with a previously classified set of data (in this case a set of patient images where the infarcted tissue has been outlined from the follow-up T2 scans). Normal distributions representing the surviving and infarcted tissue are generated from this known (or training) data, and the parameter space divided into groups. New data (or patient images) can then be classified according to this model. Each new voxel is located within the parameter space, and is classified according to the group associated with that location.

A new set of data is then used to test the quality of the model. The new data is classified according to the model ignoring for the moment the true allocation of the new data. The allocations predicted by the model are then compared with the true allocations.

In one example of the method of this invention, probability distributions were initially calculated from the data of ten patients. To validate the method, the model was then applied to seven new patients. Each patient in the training data cohort was then considered individually. A model was determined from the remaining nine patients and applied to the 10^{th} patient. The efficiency of prediction was given by measures of sensitivity, specificity, positive predictive value and negative predictive value.

It was found that an 8 dimensional model utilising both ratio (ra) and absolute measures, namely raDWI, DWI, raMTT, MTT, raCBF, CBF, raCBV and CBV gave optimal predictive efficiency. Independent use of only ratio or absolute diffusion and perfusion values significantly reduced the measures of sensitivity and positive predictive value. The mean measures of sensitivity, specificity, positive predictive value and negative predictive value for the training data sets were 0.74±0.08, 0.97±0.02, 0.68±0.09 and 0.98±0.01, respectively. For the validation data sets the values were 0.72±0.05, 0.97±0.02, 0.68±0.07 and 0.97±0.02, respectively.

A more detailed description of the above example is given in Annexure A.

The method of this invention can be implemented in computer software to provide an automated predictive model. There are four aspects which enable automation of this method, namely (i) registration of perfusion and diffusion images, (ii) mid-plane algorithm (to generate difference diffusion and MTT maps for extraction regions of tissue-at-risk of infarction and calculation of ratio diffusion and perfusion measures, (iii) 3D region growing method to extract the regions of tissue-at-risk of infarction and (iv) the parametric normal classifier algorithm to predict infarct growth.

The foregoing describes only one embodiment of the invention, and modifications which are obvious to those skilled in the art may be made thereto without departing from the scope of the invention.

For example, a modification to the methodology is the implementation of a 3D spatially-assisted parametric normal classifier algorithm to predict infarct evolution. This may increase the accuracy of the classification algorithm. Further, although the described methodology models the diffusion and perfusion metric distributions using a single Gaussian function for each group (infarcted and surviving tissue), a possible modification is to model each distribution by a mixture of Gaussian functions. This would allow more freedom for the shape of the distributions.

### REFERENCES

1. Warch S, Chien D, Li W, Ronthal M, Edelman R R. Fast magnetic resonance diffusion weighted imaging of acute human stroke. Neurology 1992;42:1717-1723.

2. Basser P J, Pierpaoli C J. Microstructural and physiological features of tissue elucidated by quantitative diffusion tensor MRI. J. Magn. Reson. [B] 1996;19:209-219.

3. Sorensen A G, Buonanno F S, Gonzalez R G, Schwamm L H, Lev M H, Huang-Hellinger F R, Reese T G, Weisskoff R M, Davis T L, Suwanwela N, Can U, Moreira J A, Copen W A, Look R B, Finklestein S P, Rosen B R, Koroshetz W J. Hyperacute stroke: evaluation with combined multisection diffusion-weighted and hemodyamically weighted echo-planar MR imaging. Radiology 1996;199:391-401.

4. Moseley M E, Cohen Y, Mintorovitch J, Chileuitt L, Shimizu H, Kucharczyk J, Wendland M F, Weinstein P R, Early detection of regional cerebral schema in cats: Comparison of diffusion- and T2- weighted MRI and spectroscopy. Magn. Reson. Med. 1990;14:330-346.

5. Moonen C T, Pekar J, De Vleeschouwer M H M, Van Gelderen P, Van Ziji P C M, Despres D. Restricted and anisotropic displacement of water in healthy cat brain and in stroke studied by NMR diffusion imaging. Magn. Reson. Med. 1991;19:327-332.

6. Welch K M A, Windham J, Knight R A, Nagesh V, Hugg J W, Jacobs M, Peck D, Booker P, Dereski M O, Levine S R. A model to predict the histopathology of human stroke using diffusion and T2 weighted magnetic resonance imaging. Stroke 1995;26:1983-1989.

7. stergaard L, Weisskoff R M, Chesler D A, Gyldensted C, Rosen B R. High resolution measurement of cerebral blood flow using intravascular tracer bolus passages, part 1: mathematical approach and statistical analysis. Magn. Reson. Med. 1996;36:715-725.

8. stergaard L, Sorensen A G, Kwong K K, Weisskoff R M, Gyldensted C, Rosen B R. High resolution measurement of cerebral blood flow using intravascular tracer bolus passages, part 2: experimental comparison and preliminary results. Magn. Reson. Med. 1996;36:726-736.

9. stergaard L, Smith D F, Vestergaard-Poulsen P, Hansen S B, Gee A D, Gjedde A, Gyidensted C. J. Cereb. Blood Flow Metab. 1998;18:425-432.

10. Collins D L, Neelin P, Peters T M, Evans A C. Automated 3-D intersubject registration of MR volumetric data in standard Talairach space. J. Comput. Assis. Tomogr. 1994;18:192-205.

11. Russ J C, The image processing handbook. (3rd ed.) Boca Ralon, Fla. 1999.

12. McLachlan G J, Basford K E. Mixture models: inference and applications to clustering. Ney York: Marcel Dekker, 1998.

### ANNEXURE A

Patients

Nineteen patients (9 male and 10 female, age 75.6±9.1) with acute focal neurologic symptoms consistent with hemispheric ischemic stroke and radiographic evidence of a diffusion-perfusion mismatch were recruited into this study. In this group of patients, the diffusion lesion normally expands into the surrounding hypoperfused territory.

The “time of first scan” was defined as the time elapsed between the initial MRI scans and the last time the patient was known to be without neurological deficit. The mean “time of first scan” was 8.9 (±3.5) hours. Five patients (9-12,16) were scanned within the six-hour window where therapeutic intervention is normally contemplated. Patients were excluded if they had cerebral hemorrhage or some other preexisting nonischemic neurological condition that would confound clinical or MR assessment. Patients enrolled in this study received serial diffusion weighted imaging (DWI) and perfusion imaging (PI) examinations. For each patient the last MRI scan was used to determine the final lesion volume. The mean last follow-up examination time was 818 (±674) hours. Three patients (1,10,12) died within seven days of onset of symptoms. In these patients, the presence of edema may result in an overestimation of final lesion volume. Patients who were treated with recombinant tissue plasminogen activator or any neuroprotective therapy were excluded from the study.

Imaging Protocol

In the acute stage, all patients received a DTI and PI scan. The single shot diffusion scan was always acquired preceding the perfusion scan. In addition, a MR angiographic (MRA) examination was performed at the initial time point to fully characterise any perfusion abnormality. All images were obtained using a 1.5 T General Electric Medical systems (GEMS) Echospeed scanner with a maximum gradient strength of 23 mT/m. Due to individual working practices at each stroke clinic and upgrades of respective echoplanar imaging (EPI) protocols, three different DTI sequences were employed. Conventional fast spin echo T2-weighted images were acquired at all time points. The total MRI examination time was within 20 minutes.

Diffusion Tensor Imaging (DTI).

Diffusion images for patients 1-9,18,19 were acquired with a spin-echo, echoplanar DTI sequence with the following acquisition, 18 axial slice full brain coverage, FOV=30 cm, TR=10s, TE=105ms, 5 mm slice thickness with 1 mm gap and 4 b-values per direction (6 gradient directions). The maximum b-value was 875 s mm⁻². The acquisition matrix was 128×144 (fractional Ky sampling) with a resulting image matrix of 256×256. Raw images were corrected for the presence of eddy current - induced warping artifacts. For patients 10-12, an optimized DTI sequence was employed. Imaging parameters were 18 axial slices, FOV=24 cm, TR=6s, TE=122 ms, 5 mm slice thickness with 1 mm gap and 28 b-values per direction [7 gradient directions, 25 high (b=1112 s mm⁻²) and 3 low b-values (b=0)]. The acquisition matrix was 96×96 and the reconstruction matrix was 128×128. For patients 13-17, the imaging parameters for the DTI sequence were 15 axial slices, FOV=24 cm, TR=10 s, TE=120 ms, 5 mm slice thickness with 1.5 mm gap and 28 b-values per direction [7 gradient directions, 21 high (max 1220 s mm⁻²) and 7 low b-values (b=0)]. The acquisition matrix was 96×96 and the reconstruction matrix was 128×128. Isotropic diffusion weighted images were derived from the trace of the diffusion tensor as reported by Sorensen et al.³

Perfusion Imaging (PI)

Quantitative cerebral blood perfusion maps were obtained utilizing dynamic fast bolus tracking of GdDTPA (30 ml, Gd-diethylenetriaminepenta acetate “Magnevist”, Schering, Germany) using a spin echo EPI sequence. The imaging parameters were for patients 1-9,18,19: 10 axial slices, FOV=30 cm, image matrix=128×128, TR=1.85 s, TE=60 ms, 7 mm slice thickness with 1 mm gap with acquisition of 50 frames per slice. For patients 10-12, 13 axial slices were acquired with FOV=24 cm, image matrix 128×128, TR=2.51 s, TE=60 ms, 7 mm slice thickness with 1 mm gap with acquisition of 30 frames per slice. The imaging parameters for patients (13-17) were: 9 axial slices, FOV=24 cm, TR=1.85 s, TE=60 ms, 7 mm slice with 2.5 mm gap with acquisition of 50 frames per slice. Baseline images were acquired for a period of 10 s, after which the contrast agent was injected with a Medrad Power Injector at 5 ml s⁻¹. Quantitative maps of CBF, CBV and MTT were calculated using the method described by Ostergaard et al. To cover the entire penumbral territory the perfusion images were acquired with an increased slice thickness and slice gap compared to the DTI sequence. The perfusion maps were subsequently registered and re-sliced to the initially prescribed diffusion images using the methods described below.

Image Processing

Image Registration and Calculation of Diffusion and Perfusion Metrics

Apart from the manual definition of a rectangular ROI around the MCA (via mouse control), an automated algorithm was used to define the optimum arterial input function prior to calculation of CBF maps. For every pixel within the described ROI a cubic spline was evaluated to model pixel signal as a function of time. The cubic spline function possessing the largest minima and the least signal fluctuation was then selected. The corresponding pixel was assigned as the MCA pixel which best represented the arterial input function. In this study, CBF maps were generated from arterial input functions defined from the MCA contralateral to the DWI lesion. Subsequent ultrasound evaluation of the carotid artery on the side used for the arterial input function did not reveal any stenosis greater than 50%. To enable registration of perfusion maps to diffusion images, raw spin-echo EPI perfusion images were coregistered to the initial T2-weighted DWI scan (b=0) using a 6 parameter rigid body transformationa.¹⁰ A similar transformation was also used to coregister serial diffusion scans. The final lesion volume was derived after normalisation and subtraction of initial T2-weighted diffusion scans (b=0) from follow-up DTI scans (b=0). This enabled a more accurate delineation of the infarct volume as pixels with hyperintense signal originating from ventricular and sulcal cerebral spinal fluid (CSF) were excluded from the T2 lesion mask. An automated mid-plane algorithm was used to calculate diffusion and perfusion ratio measures between the infarcted and contralateral hemispheres. This algorithm comprised flipping the image in the Y plane followed by registration of the mirrored image to it's original form with a six parameter rigid body transformation. The mid-plane was then determined by halving the resulting rotations and translations. Difference maps (eg. dDWI and dMTT) for the ischemic territory were generated by the subtraction of corresponding voxels in the contralateral hemisphere. To aid delineation of the diffusion abnormality on the initial isotropically weighted diffusion image³, a composite image was calculated from the product of the initial diffusion image and the dDWI map on a pixel-by-pixel basis. A bimodal t-test was then performed on this image to create a binary diffusion mask. In a similar fashion, a MTT composite image was calculated by multiplication of the initial MTT map with the dMTT map and with the initial isotropically weighted diffusion image on a pixel-by-pixel basis. This yielded a MTT mask that was specific only to brain tissue as defined on the DWI scan. Using the DWI mask as the initial seed, a three dimensional region-growing technique¹¹ was then employed to extract the MTT mask from the composite MTT map. The task of extracting the MTT mask was simplified by only interrogating the hemisphere containing the ischemic lesion. Intermediate composite maps along with binary diffusion and MTT masks for a representative patient (subject 5) are given in FIG. 1. Absolute and ratio perfusion measures between the infarcted and contralateral hemispheres for three specific penumbral regions were interrogated. These three regions were the initial diffusion ROI, the territories within the MTT mask that went onto infarction and the tissue that survived the ischemic episode. Differences between perfusion measures for the three regions were tested with ANOVA.

Parametric Normal Classifiers

Parametric normal classifiers were employed to predict the spatial location and size of the final lesion from diffusion and perfusion images acquired in the acute stage of stroke. Each pixel in the model was classified into two groups: those corresponding to the final T2 lesion, which are defined as infarcted, and those representing tissue that has survived the ischemic event. For the purpose of illustration, a model employing a parameter vector×containing the diffusion and MTT pixel intensities was defined (see FIG. 2). Representative frequency histograms were produced where the isotropically weighted diffusion pixel intensity (arbitrary units) is plotted versus MTT measures for all pixels outside (histogram A, pixels colour coded blue) and within the final lesion volume (histogram B, pixels colour coded red). In 2(C), each histogram bin is classified into one of the two groups in accordance with the frequencies in histograms A and B. Mathematically, each group can be modeled by a normal distribution (ƒᵢ) with a mean parameter vector (μᵢ, containing d parameters, covariance matrix (Σᵢ) and prior probability (pᵢ) determined from the training data set using the following equation,

$${f}_{ i} ( x ) = \frac{{p}_{i}}{{( 2 π )}^{d / 2} {\vert {\sum }_{i} \vert}^{1 / 2}} exp ( - \frac{1}{2} {( x - {μ}_{i} )}^{T} {\sum }_{i}^{- 1} ( x - {μ}_{i} ) ) \qquad ( 1 )$$

The histograms A and B given in FIG. 2, were modeled by the normal distributions in histograms D and E, respectively. The model classifies each new pixel in accordance with the relative heights of the two group allocation functions:

$$g ( x ) = arg {max}_{i} {f}_{ i} ( x ) \qquad ( 2 )$$

2(F) shows the resultant classification function. New pixels that fall within the red region would be allocated as destined to infarct, whilst those in the blue would be assigned as penumbral tissue that would survive the ischemic event. This methodology was employed using an eight-parameter vector (DWI, rₐDWI, CBF, rₐCBF, CBV, rₐCBV, MTT and rₐMTT). Probability distributions were initially calculated from the data of ten patients (subjects 1-10). To validate the method, the model was then applied to seven novel patients (11-17). Each patient in the training data cohort was then considered individually. A model was determined from the remaining nine patients and applied to the individual patient. The efficiency of prediction was given by measures of sensitivity, specificity, positive predictive value and negative predictive value.³⁵

Results

Patient demographic and imaging data are given in Table 1. Mean volumes of the automatically extracted diffusion lesion and MTT mask measured at the initial time point were 17.4±21.7 and 69.0±65.3 ml respectively. The mean follow-up final lesion volume measured from the T2 weighted DTI scan (b=0) was 64.6±59.5 ml. Perfusion measures derived from the automatically extracted masks are listed in Table 2. The mean rₐCBF and rₐCBV values for the ROI defined by the corresponding initial DWI lesion were 0.54±0.19 and 1.02±0.30. The mean rₐCBF and rₐCBV values for the entire infarcted territory within the MTT mask were 0.70±0.19 and 1.20±0.36. For recovered tissue within the MTT mask, the mean rₐCBF and rₐCBV values were 0.99±0.25 and 1.87±0.71 respectively. There was a significant difference between the initial diffusion ROI and recovered MTT territory for both of these perfusion measures (both p <0.0001). Comparison of the mean rₐCBF and rₐCBV values for tissue within the infarcted and recovered MTT masked territory also revealed significant differences between the two regions. The level of significance for the two measures were p <0.003 and p <0.001, respectively. As expected, the MTT territory that survived infarction exhibited the largest rₐCBF values.

For absolute perfusion measures, the mean CBF (ml/100 g/min) and CBV (ml/100g) values for the corresponding initial DWI lesion were 26.6±8.3 and 3.4±1.2. The mean CBF and CBV values for the total infarcted territory were 33.9±9.7and 4.2±1.9. For recovered tissue within the MTT mask, the mean CBF and CBV values were 41.5±7.2 and 5.3±1.2, respectively. For normal tissue, defined as tissue within the MTT mask reflected onto the contralateral hemisphere, the CBF and CBV values were 58.6±14.7 (ml/100 g/min) and 4.2±1.4 (ml/100 g/min), respectively. These values correlate to previously reported perfusion measures. There was a significant difference between the initial diffusion ROI and recovered tissue within the MTT mask for both absolute perfusion measures (p <0.0001). A significant difference was also found for the CBF and CBV values in the infarcted and recovered MTT regions, p <0.009 and p <0.036 respectively. The significance level for the difference in CBF values for normal and recovered tissue was p <0.0001. In contrast, the cerebral blood volume was increased in this important penumbral territory (p <0.011). Hypervolemia in the ischemic penumbra, measured using MR dynamic bolus tracking has previously been reported.

Measures of efficiency of the predictive model are given in Table 1. It was found that an 8 dimensional model utilising the metrics rₐDWI, DWI, rₐMTT, MTT, rₐCBF, CBF, rₐCBV and CBV gave optimal predictive efficiency. Independent use of only ratio or absolute diffusion and perfusion values, significantly reduced the measures of sensitivity and positive predictive value. The mean measures of sensitivity, specificity, positive predictive value and negative predictive value for the training data sets (patients 1-10) were 0.74±0.08, 0.97±0.02, 0.68±0.09 and 0.98±0.01, respectively. For the validation data sets (patients 11-17) the values were 0.72±0.05, 0.97±0.02, 0.68±0.07 and 0.97±0.02, respectively. The measures of predictive efficiency including results for both subjects (18,19) who presented with progressive occlusion of the MCA, found on serial MRA examinations, were 0.65±0.17, 0.96±0.04, 0.63±0.12 and 0.96±0.04, respectively. The measures of predictive efficiency for the five subjects (9-12,16) scanned within six hours of onset of symptoms were 0.73±0.06, 0.96±0.02, 0.69±0.05 and 0.97±0.02, respectively.

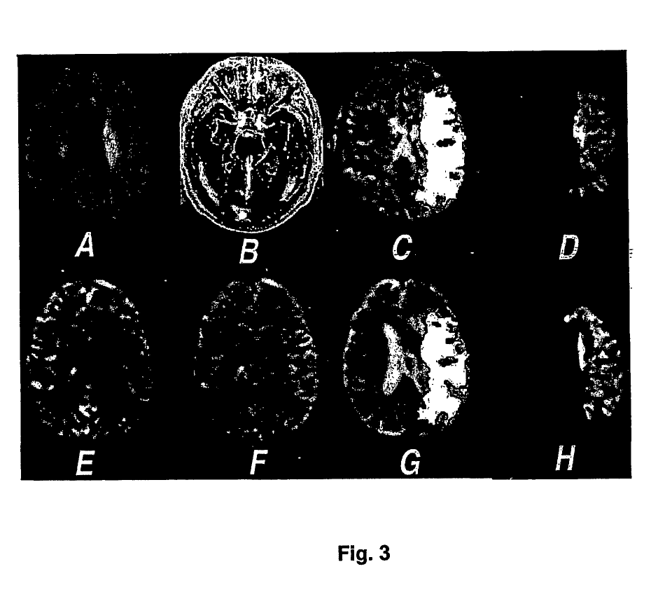

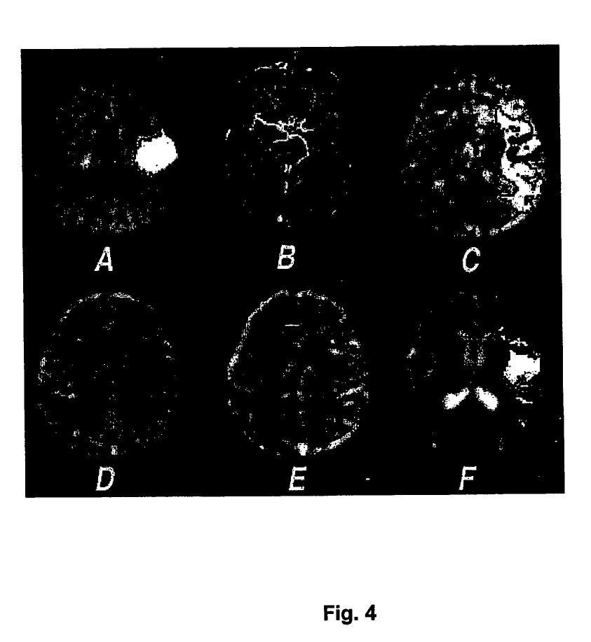

Diffusion and perfusion maps together with predicted infarct territories for two representative patients are given in FIGS. 3 and 4. These images show an arbitrary mid-stroke slice for patients belonging to the training data cohort (patient 10, FIG. 3) and validation data set (patient 17, FIG. 4), respectively. The extracted MTT masks are coloured blue with the corresponding predicted infarct territory coloured red. For patient 10, 2 hours after onset of symptoms (FIG. 3), the MRA shows an occlusion of the left MCA along with a small, poorly defined diffusion lesion in deep white matter of the MCA territory with a corresponding large MTT abnormality. The MTT map revealed areas of reduced CBF and increased CBV. Although there is some evidence of edema on the follow-up T2 weighted image, there is a close correlation between the predicted lesion size and the T2 defined infarct volume. In this case the model correctly predicted that the infarct would grow into the entire hypoperfused territory even though the MTT region contained predominantly hypervolemic tissue. The images of patient 17 shown in FIG. 4, acquired 10 hours after onset of symptoms, reveal a well-defined DWI lesion resulting from occlusion of the left MCA. The large MTT abnormality shows regions of reduced CBF and a heterogeneous pattern of both reduced and elevated CBV. In this case, the model correctly predicted that the infarct would not evolve in size beyond the initial DWI lesion. As can be seen in Table 1, the volume of the extracted MTT masks for patients in this study correlated with the final lesion volume (r=0.88). The mean MTT mask and final lesion volumes were 69±65.3 and 64.6±59.5 ml. This correlation demonstrates that for this group of subjects the extracted masks correctly identified tissue with an altered hemodynamic function. The computational time, including calculation and registration of DWI and PI maps and modeling of infarct evolution was less than 10 minutes using a Silicon Graphics Octane workstation.

Discussion

This example used a strategy to automatically extract masks of the diffusion lesion and regions of abnormal hemodynamic function defined on MTT maps acquired in the acute stage of stroke. This methodology allows rapid assessment of diffusion, CBF, CBV and MTT measures within the MTT mask, including the diffusion—perfusion mismatch and estimation of infarct evolution using predictive modeling techniques. Recent studies have redefined the relationships between the ischemic penumbra and diffusion and perfusion abnormalities seen on MR imaging. The predictive modeling strategy reported in this study does not depend upon the identification of an ischemic penumbra. This methodology may prove useful for patient assessment prior to possible therapeutic intervention and importantly in the analysis of data from large clinical stroke trials.

Surprisingly few studies have been published in the literature reporting MR-derived perfusion measures within the penumbral territory in humans. Many of these studies have relied on the use of manually defined ROIs on perfusion images and therefore contain additional information from non-brain tissue from ventricular or sulcal regions. The data obtained in this example extends previous results by including absolute measures of blood flow and blood volume in the MTT territory from both infarcted tissue and tissue which survived the ischemic event.

In the territory of the MTT mask, a significant decrease in rₐCBF (0.70±0.19) and CBF (33.9±9.7 ml/100 g/min) was found in tissue that went onto infarction compared with tissue which survived the ischemic event (0.99±0.25 and 41.5±7.2 ml/100 g/min, respectively). The rₐCBF values calculated in our study are very similar to those reported from SPECT studies namely, 0.48±0.10 and 0.75±0.10 for the ischemic core and penumbra respectively. Quantitative CBF measures in the the initial DWI lesion and diffusion-perfusion mismatch territory of 34.4±22.4 and 50.2±17.5 (ml/100 g/min) have been reported in stroke patients. Although these values are similar to those measured in this example, no distinction was made in that earlier study between tissue that survived or went onto infarction in the MTT territory. In the group of patients investigated in this example, the CBF was reduced in all regions of the MTT territory compared with normal tissue on the contralateral side. This included tissue within the MTT mask that recovered or eventually progressed to infarction. An analogous result has been reported previously. In five of the nineteen patients, there was increased CBF in the diffusion—perfusion mismatch region which progressed to infarction, as defined on the follow-up T2 weighted scan (patients 6,7 10,14 and 19). In this example, this observation was not apparent in contralateral ratio measures.

This finding highlights an advantage of measuring absolute rather than ratio perfusion measures within the MTT ROI. The accuracy of ratio measures relies on a number of factors. These include (i) symmetrical brain morphology, (ii) the bilateral absence of pathological processes such as white matter disease, and (iii) head positioning in the scanner so that the brain appears symmetrical in the sagittal plane. Although the underlying pathophysiological reason for this observation is unclear, a possible mechanism may involve collateral flow to leptomeningeal vessels already undergoing vasodilation due to an altered hemodynamic function or a process involving increased flow via anastamotic vessels to a hypoperfused region. The finding of increased penumbral blood flow has been reported by others using both ratio measures and quantitative arterial spin labeling methods. The diffusion—perfusion mismatch regions with increased CBF correlated with tissue exhibiting enhanced CBV. Such a correlation gives evidence of a possible mechanism involving vasodilation of collateral leptomeningeal vessels. This highlights the fact that within the MTT territory, tissue that survives the ischemic event is not always restricted to regions with increased cerebral blood flow.

Patients in this example exhibited a heterogeneous pattern of both reduced and elevated cerebral blood volume measures within the MTT mask. Penumbral tissue with increased measures of CBV have been reported in other studies. Elevated CBV measures have been shown not to result from a breakdown of the blood-brain barrier and leakage of Gd-DTPA but to vasodilation of leptomeningeal vessels in response to an altered hemodynamic state to maintain cerebral perfusion pressure. Due to the diverse nature of CBV values in the MTT mask in the present study, predicting infarct evolution utilising threshold levels of this metric may have limited use. In humans, the modelling of stoke evolution is a complex problem because of the limited information that can be obtained in vivo regarding some of the important underlying mechanisms believed to be involved with neuronal death. Thus diffusion and perfusion imaging are used as surrogate markers to model and predict complex pathophysiological processes such as apoptosis, that occur following an ischemic episode. However, given these constraints, it has been demonstrated that diffusion and perfusion measures acquired in the acute phase of stroke can be used to model infarct evolution.

Although the time of first scan after onset of symptoms was 8.9±3.5 hours, it was found that exclusion of the diffusion metrics did not reduce the model's predictive power. The measures of sensitivity, specificity, positive predictive value and negative predictive value for the validation data sets derived using only the perfusion metrics were 0.72±0.05, 0.97±0.02, 0.67±0.07 and 0.97±0.02, respectively. Furthermore, for the five patients (9-12,16) who were scanned within the six hour window after onset of symptoms the measures of predictive efficiency were of similar magnitude, namely 0.73±0.06, 0.96±0.02, 0.69±0.05 and 0.97±0.02, respectively. This suggests that this methodology may be suitable for hyperacute stroke patients (<6 hours after onset of symptoms) which present with large diffusion-perfusion mismatches. With this strategy, it is assumed that the MTT mask represents the boundary for possible infarct evolution. It is possible with this methodology for the predicted lesion to be slightly larger than the calculated MTT mask. Such a result can be seen in three patient's data (see Table 1, patients 4,7,19). This anomaly can arise when the diffusion mask is not spatially congruent with the MTT mask, i.e. a portion of the diffusion mask lays outside of the MTT masked region. This problem can occur when registration of the diffusion and perfusion images is comprimised because of head movement or the presence of artifacts within the diffusion image. In this example, the contralateral MCA was routinely..used to define the arterial input function for the calculation of perfusion maps. In using this vessel, it is assumed that there is little or no concurrent carotid stenosis or occlusion that may affect the accuracy of resulting perfusion maps. Two patients (5,10) possessed moderate contralateral stenoic carotid arteries (50-75%) and one (19) had significant occtusion (80-90%). Although the predictive model was accurate for both patients (5,10), further work may fully determine the correlation between concurrent carotid stenosis and model efficiency. In addition, a larger subject cohort may also enable identification of distinctive angiographic and perfusion characteristics that allow recognition of acute stroke patients who present with progressive occlusion of the MCA.

TABLE 1
Summary of Imaging Results

| | | | | | | | | Measure of Accuracy | | | |
|---|---|---|---|---|---|---|---|---|---|---|---|
| | | | | | | | Predicted | | | Positive | Negative |
| | Arterial | Time of first | Acute volumes (ml) | | | Follow-up | volume | | | Predictive | Predictive |
| Patient | Territory | scan (hrs) | DWI | MTT | mismatch | T2 (hrs) | (ml) | Sensitivity | Specificity | value | value |
| 1 | MCA + PCA | 12 | 45.0 | 77.9 | 32.9 | 70.0 (111) | 74.5 | 0.76 | 0.97 | 0.71 | 0.98 |
| 2 | MCA_sv | 13 | 6.3 | 14.7 | 8.4 | 11.5 (1290) | 13.7 | 0.85 | 0.99 | 0.71 | 0.99 |
| 3 | MCA_sv | 13 | 13.0 | 15.6 | 2.6 | 14.9 (910) | 15.4 | 0.66 | 0.99 | 0.64 | 0.99 |
| 4 | MCA_sv | 8 | 8.2 | 46.5 | 38.3 | 36.1 (749) | 48.4 | 0.75 | 0.96 | 0.56 | 0.98 |
| 5 | MCA_sv | 11 | 10.8 | 28.9 | 18.1 | 27.8 (827) | 28.8 | 0.75 | 0.98 | 0.74 | 0.98 |
| 6 | MCA | 13 | 9.5 | 48.5 | 39.0 | 56.9 (2160) | 47.5 | 0.59 | 0.98 | 0.71 | 0.96 |
| 7 | MCA_sv | 12 | 3.3 | 34.6 | 31.3 | 23.1 (182) | 37.7 | 0.83 | 0.94 | 0.51 | 0.99 |
| 8 | MCA_sv | 12 | 4.1 | 8.8 | 4.7 | 8.5 (2688) | 8.2 | 0.69 | 0.99 | 0.80 | 0.99 |
| 9 | MCA_sv | 6 | 2.1 | 8.3 | 6.2 | 7.6 (1176) | 8.2 | 0.80 | 0.99 | 0.74 | 0.99 |
| 10 | MCA | 2 | 2.7 | 169.5 | 166.8 | 167.1 (96) | 168.8 | 0.69 | 0.95 | 0.69 | 0.95 |
| 11 | MCA | 4 | 70.9 | 168.9 | 98.0 | 148.9 (724) | 163.4 | 0.80 | 0.95 | 0.73 | 0.97 |
| 12 | MCA | 3 | 25.7 | 142.7 | 117.0 | 134.2 (96) | 137.9 | 0.66 | 0.94 | 0.64 | 0.94 |
| 13 | MCA | 7 | 75.9 | 146.0 | 70.1 | 146.2 (806) | 137.8 | 0.69 | 0.95 | 0.73 | 0.94 |
| 14 | MCA_sv | 9 | 0.4 | 6.4 | 4.0 | 5.0 (678) | 4.4 | 0.68 | 0.99 | 0.79 | 0.99 |
| 15 | PCA | 11 | 7.9 | 23.8 | 15.9 | 20.6 (691) | 22.7 | 0.73 | 0.98 | 0.66 | 0.99 |
| 16 | PCA | 6 | 8.2 | 20.9 | 12.7 | 16.9 (720) | 18.6 | 0.72 | 0.99 | 0.65 | 0.99 |
| 17 | MCA | 10 | 19.3 | 60.2 | 40.9 | 42.2 (738) | 55.8 | 0.76 | 0.98 | 0.58 | 0.99 |
| 18* | MCA | 10 | 13.6 | 210.0 | 196.4 | 117.8 (745) | 199.4 | 0.71 | 0.88 | 0.42 | 0.96 |
| 19* | MCA | 7 | 3.5 | 79.2 | 75.7 | 171.6 (151) | 82.8 | 0.24 | 0.95 | 0.50 | 0.87 |
| mean (1-10) | | | | | | | | 0.74 | 0.97 | 0.68 | 0.98 |
| SD | | | | | | | | 0.08 | 0.02 | 0.09 | 0.01 |
| mean (12-17) | | | | | | | | 0.72 | 0.97 | 0.68 | 0.97 |
| SD | | | | | | | | 0.05 | 0.02 | 0.07 | [symbol] |
| mean (12-19) | | | | | | | | 0.65 | 0.96 | 0.63 | 0.96 |
| SD | | | | | | | | 0.17 | 0.04 | 0.12 | 0.04 |
| mean (9-12, 16) | | | | | | | | 0.73 | 0.96 | 0.69 | 0.97 |
| SD | | | | | | | | 0.06 | 0.02 | 0.05 | 0.02 |

[symbol]

Serial MRA examinations revealed progressive occlusion of the MCA. MVA-sv denotes small vessel occlusion in the MCA territory

TABLE 2
Summary of Perfusion Imaging Results

| | rₐCBF | | | CBF (ml/100 g/min) | | | | rₐCBV | | | CBV (ml/100 g) | | | |
|---|---|---|---|---|---|---|---|---|---|---|---|---|---|---|
| | initial | | | initial | | | nor- | initial | | | initial | | | nor- |
| | DWI | infarcted | recovered | DWI | infarcted | recovered | mal | DWI | infarcted | recovered | DWI | infarcted | recovered | mal |
| Patient | lesion | tissue | tissue | lesion | tissue | tissue | tissue | lesion | tissue | tissue | lesion | tissue | tissue | tissue |
| 1 | 0.45 | 0.64 | 1.36 | 22.7 | 27.4 | 43.9 | 59.5 | 0.79 | 0.84 | 2.03 | 2.1 | 2.2 | 4.9 | 3.7 |
| 2 | 0.78 | 0.73 | 1.30 | 20.8 | 21.8 | 34.2 | 39.1 | 1.34 | 1.07 | 1.74 | 2.6 | 2.5 | 3.6 | 3.2 |
| 3 | 0.63 | 0.77 | 1.03 | 26.3 | 34.2 | 44.9 | 49.2 | 0.89 | 0.88 | 1.31 | 2.5 | 2.8 | 4.4 | 3.7 |
| 4 | 0.43 | 0.68 | 0.88 | 20.2 | 34.5 | 42.3 | 55.9 | 1.29 | 1.89 | 2.24 | 3.5 | 5.6 | 6.8 | 3.3 |
| 5 | 0.41 | 0.62 | 1.23 | 33.3 | 43.0 | 46.0 | 83.2 | 0.94 | 1.07 | 1.75 | 4.6 | 5.0 | 6.3 | [symbol] |
| 6 | 0.73 | 0.99 | 1.10 | 26.5 | 32.2 | 30.2 | 39.6 | 1.42 | 1.77 | 2.80 | 4.9 | 6.1 | 6.1 | 3.9 |
| 7 | 0.64 | 0.77 | 0.95 | 39.2 | 52.9 | 48.2 | 76.6 | 1.29 | 1.63 | 1.73 | 5.5 | 8.0 | 6.3 | 5.4 |
| 8 | 0.69 | 0.87 | 1.15 | 33.5 | 38.0 | 47.5 | 53.0 | 1.11 | 1.21 | 1.42 | 3.7 | 3.6 | 3.9 | 3.7 |
| 9 | 0.79 | 0.84 | 0.92 | 23.9 | 27.2 | 32.0 | 38.2 | 1.38 | 1.37 | 1.31 | 3.0 | 3.5 | 2.8 | 3.0 |
| 10 | 0.39 | 0.71 | 0.71 | 28.6 | 35.3 | 34.3 | 59.0 | 0.72 | 1.51 | 1.54 | 3.3 | 4.3 | 4.2 | 3.2 |
| 11 | 0.33 | 0.48 | 0.94 | 18.7 | 24.7 | 48.6 | 58.0 | 0.67 | 0.86 | 1.79 | 2.5 | 3.1 | 6.8 | 4.0 |
| 12 | 0.26 | 0.60 | 0.81 | 13.9 | 23.1 | 33.2 | 51.8 | 0.58 | 1.12 | 1.78 | 2.4 | 3.7 | 5.8 | 4.0 |
| 13 | 0.23 | 0.34 | 0.56 | 16.2 | 24.1 | 38.1 | 79.0 | 0.46 | 0.57 | 1.38 | 1.9 | 2.6 | 6.1 | 5.1 |
| 14 | 0.58 | 0.67 | 0.73 | 34.7 | 49.8 | 45.8 | 79.8 | 0.78 | 1.07 | 1.06 | 5.9 | 9.0 | 6.8 | 8.6 |
| 15 | 0.56 | 0.46 | 0.64 | 24.4 | 26.3 | 40.1 | 71.4 | 0.91 | 0.75 | 1.07 | 2.7 | 3.2 | 5.5 | 6.0 |
| 16 | 0.45 | 0.64 | 1.35 | 15.9 | 25.8 | 31.8 | 39.3 | 0.91 | 1.02 | 4.15 | 2.4 | 2.8 | 4.2 | 2.8 |
| 17 | 0.39 | 0.49 | 0.81 | 26.3 | 32.8 | 54.7 | 71.5 | 1.31 | 1.41 | 2.48 | 2.7 | 3.1 | 6.1 | 2.5 |
| 18 | 0.80 | 1.00 | 1.18 | 43.1 | 39.8 | 44.4 | 49.5 | 1.36 | 1.57 | 2.07 | 4.9 | 4.5 | 4.9 | 3.5 |
| 19 | 0.79 | 1.05 | 1.25 | 37.2 | 50.9 | 48.8 | 59.3 | 1.16 | 1.24 | 1.94 | 3.6 | 4.4 | 5.5 | 4.0 |
| mean | 0.54 | 0.70 | 0.99 | 26.6 | 33.9 | 41.5 | 58.6 | 1.02 | 1.20 | 1.87 | 3.4 | 4.2 | 5.3 | 4.2 |
| SD | 0.19 | 0.19 | 0.25 | 8.3 | 9.7 | 7.2 | 14.7 | 0.30 | 0.36 | 0.71 | 1.2 | 1.9 | 1.2 | 1.4 |

Note:
infarcted tissue represents brain tissue within the MTT mask that went onto infarction and recovered tissue represents tissue within the MTT mask that survived the ischemic event

1. A method of predicting deterioration of cerebral tissue of a patient due to a stroke, the method including the steps of:
processing diffusion and perfusion images of the cerebral tissue obtained by magnetic resonance imaging shortly after the onset of stroke symptoms, to automatically define regions of interest on the images and to calculate diffusion and perfusion ratio measures, and
identifying pixels in the regions of interest representing tissue expected to go into infarction, by applying a classifier algorithm which uses a plurality of parameters including the calculated diffusion and perfusion ratio measures.

2. A method as claimed in claim 1, wherein the images include an isotropically weighted..diffusion image.

3. A method as claimed in claim 1, wherein the perfusion images include one or more maps of cerebral blood flow, cerebral blood volume and mean transit time.

4. A method as claimed in claim 3, further comprising the step of registering the diffusion and perfusion images before processing the images.

5. A method as claimed in claim 2, wherein the processing step includes using a mid-plane algorithm to generate at least one difference diffusion weighted image and at least one difference perfusion image.

6. A method as claimed in claim 5, wherein the difference diffusion weighted image is obtained from registering the diffusion image with its mirrored image, further including the steps of forming a composite image from the product of the diffusion image and the difference diffusion weighted image on a pixel-by-pixel basis, and performing a bimodal t-test on the composite image to create a binary diffusion mask.

7. A method as claimed in claim 6, further including the step of obtaining a composite perfusion image by multiplying the perfusion image and the difference perfusion image and the diffusion image on a pixel-by-pixel basis to obtain a composite perfusion mask, and
automatically defining the region(s) of interest from the composite perfusion mask using a three-dimensional region-growing technique, using the diffusion image as an initial seed.

8. A method as claimed in claim 7, wherein the perfusion image is a map of mean transit time.

9. A method as claimed in claim 1, wherein the ratio measures are obtained by dividing the intensity of each pixel in the region(s) of interest by the corresponding pixel in the contralateral side of the associated image.

10. A method as claimed in claim 1, wherein the classifier algorithm identifies pixels representing tissue destined to go into infarction by reference to a model derived from diffusion and perfusion images from other patients.

11. A method as claimed in claim 10, wherein the classifier algorithm uses both absolute and relative values of weighted diffusion image, cerebral blood flow, cerebral blood volume and mean transit time.

12. A method as claimed in claim 1, wherein the processing and identifying steps are automated, and performed by computer software.

13. A method of predicting evolutionary effects of a stroke on cerebral tissue of a patient, including the steps of
digitally processing magnetic resonance diffusion and perfusion images of the cerebral tissue of the patient obtained during an early stage of the stroke, to thereby identify regions of interest at risk of infarction and to calculate modelling parameter values from the images, and
automatically identifying image pixels representing cerebral tissue expected to go into infarction, by applying an algorithm using the calculated modelling parameter values.

14. A method as claimed in claim 13, wherein the processing step includes automatic identification of regions of interest in the images which represent tissue at risk of infarction, and wherein the identifying step is limited to pixels in the region(s) of interest.

15. A method as claimed in claim 13, wherein the modelling parameter values include ratio diffusion and perfusion measures.

16. A method as claimed in claim 13, wherein the algorithm is a parametric normal classifier algorithm using both absolute and ratio diffusion and perfusion measures.

17. A method as claimed in claim 13, wherein the image pixels representing cerebral tissue expected to go into infarction are identified by reference to a model derived from diffusion and perfusion images from other patients.

18. A method as claimed in claim 17, wherein the model includes calculating normal distributions of frequency histograms plotting pixel intensity in diffusion weighted images versus corresponding mean transit time measures for known surviving and infarcted tissue from the other patients, the method further including the step of automatically classifying each pixel in an identified region of interest for the patient by reference to the two normal distributions in the model.

19. A method as claimed in claim 13, wherein the processing and identifying steps are performed by computer software.
